(19) United States
(12) Patent Application Publication
(10) Pub. No.: US 2017/0157145 A1
(43) Pub. Date: Jun. 8, 2017

(54) SUBSTANCE INDUCING EXPRESSION OF PARKIN POLYPEPTIDE AND USE THEREOF

(71) Applicants: Samsung Electronics Co., Ltd., Suwon-si (KR); Research & Business Foundation, Sungkyunkwan University, Suwon-si (KR)

(72) Inventors: Yunil Lee, Yongin-si (KR); Yunjong Lee, Cheongju-si (KR); Sangwoo Ham, Suwon-si (KR); Sangchul Park, Seongnam-si (KR); Jooho Shin, Seoul (KR)

(21) Appl. No.: 15/332,306

(22) Filed: Oct. 24, 2016

(30) Foreign Application Priority Data
Dec. 7, 2015 (KR) 10-2015-0173296

(51) Int. Cl. A61K 31/573; G01N 33/50; C12N 9/00; A61K 31/4196; A61K 31/407

(52) U.S. Cl. CPC A61K 31/573; A61K 31/4196; A61K 31/407; C12N 9/93; C12Y 603/02; G01N 33/5023; G01N 2333/9015

(57) ABSTRACT

A method of inducing Parkin expression; inhibiting oxidative stress; and inhibiting cell death of cells by administering deferasirox, hydrocortisone, ketorolac, dexamethasone, and prednisone, a pharmaceutically acceptable salt, stereoisomer, derivative, or solvate thereof to the cell, optionally in a subject, as well as a method of preventing or treating neurodegenerative disease in the subject.

### CROSS-REFERENCE TO RELATED APPLICATION

This application claims the benefit of Korean Patent Application No. 10-2015-0173296, filed on Dec. 7, 2015, in the Korean Intellectual Property Office, the disclosure of which is incorporated herein in its entirety by reference.

### BACKGROUND

1. Field

The present disclosure relates to a pharmaceutical composition for preventing or treating neurodegenerative diseases, a pharmaceutical composition including a substance inducing expression of Parkin polypeptide, a pharmaceutical composition for inducing expression of Parkin polypeptide, a method of preventing or treating neurodegenerative diseases by using the same, and a method of inducing expression of Parkin polypeptide in a cell or in a cell of a subject.

2. Description of the Related Art

Neurodegenerative diseases are associated with conditions in which neuronal cells deteriorate, lose function, and often die. As they are generally progressive, the consequences of neurodegenerative diseases are often devastating. Patients with neurodegenerative disease may suffer severe deterioration in cognitive or motor ability. As a result, their quality of life and life expectancy may be considerably reduced.

Parkin polypeptide is an E3 ligase, and its mutations have been reported in a representative neurodegenerative movement disorder, Parkinson's disease and many cancers. Parkin polypeptide has been most actively studied in neurodegenerative diseases, and a disorder in the enzymatic activity of Parkin by mutation or oxidative stress plays an important role in mediating dopaminergic neuronal death in Parkinson's disease. It was reported that dopaminergic neuronal death is induced in Parkin depletion or functional disorder models, whereas dopaminergic neuronal death is inhibited by Parkin overexpression in oxidative stress-induced toxin mouse models. The protecting effect of Parkin against Alzheimer's toxic protein, beta-amyloid was also reported, and its protecting effect against oxidative stress in muscle cells was also reported.

Accordingly, there is a demand for a composition and a method which may be used for the prevention or treatment of neurodegenerative diseases by increasing Parkin expression.

### SUMMARY

Provided herein is a method of preventing or treating neurodegenerative disease comprising administering one or more compounds selected from the group consisting of deferasirox, hydrocortisone, and ketorolac, dexamethasone, prednisone, a pharmaceutically acceptable salt, stereoisomer, derivative, or solvate thereof, or a combination thereof.

Also provided herein is a method of inducing expression of Parkin polypeptide comprising administering one or more compounds selected from the group consisting of deferasirox, hydrocortisone, and ketorolac, dexamethasone, prednisone, a pharmaceutically acceptable salt, stereoisomer, derivative, or solvate thereof, or a combination thereof.

### BRIEF DESCRIPTION OF THE DRAWINGS

These and/or other aspects will become apparent and more readily appreciated from the following description of the exemplary embodiments, taken in conjunction with the accompanying drawing in which:

FIG. 1 is an image obtained by immunoblotting cells cultured in the presence of selected compounds using an anti-Parkin protein antibody.

FIGS. 2A-2E are provided. FIG. 2A shows schedules for treatments of hydrocortisone and/or 6-hydroxydopamine (6-OHDA) in a Parkinson's disease model. FIG. 2B is a graph showing quantification of relative parkin expression levels normalized to β-actin in the indicated brain subregions of mice (CTX, cortex; STR, striatum; VM, ventral midbrain; CB, cerebellum) treated with DMSO (vehicle) or hydrocortisone (n=3 mice per group). (*:P<0.05, **:P<0.01). FIGS. 2C and 2D are western blot analyses of parkin and its substrate AIMP2 in the striatum and ventral midbrain regions from mice treated with hydrocortisone (open markers) or DMSO (vehicle, black markers) for 7 days (*:P<0.05, **:P<0.01, and ***:P<0.001). Relative parkin or AIMP2 protein levels normalized to β-actin are shown as plot graphs (n=3 mice per group). FIG. 2E is a graph showing stereological assessment of tyrosine hydroxylase-positive dopaminergic neurons in the substantia nigra pars compacta of the indicated mouse groups (n=5 or 4 per group, **:P<0.01, and ***:P<0.001).

### DETAILED DESCRIPTION

An aspect provides a pharmaceutical composition for preventing or treating neurodegenerative diseases, the composition including one or more compounds selected from the group consisting of deferasirox, hydrocortisone, and ketorolac, dexamethasone, prednisone, a pharmaceutically acceptable salt, stereoisomer, derivative, or solvate thereof, or a combination thereof.

Deferasirox refers to 4-[(3Z,5E)-3,5-bis(6-oxo-1-cyclohexa-2,4-dienylidene)-1,2,4-triazolidin-1-yl] benzoic acid. Deferasirox is known as an iron chelator. Deferasirox may be administered orally.

The term “hydrocortisone” may be used interchangeably with “cortisol”. Hydrocortisone refers to ( 11β)-11,17,21-trihydroxypregn-4-ene-3,20-dione. Hydrocortisone may be administered orally, intravenously, topically, or rectally.

Ketorolac refers to (±)-5-benzoyl-2,3-dihydro-1H-pyrrolizine-1-carboxylic acid. Ketorolac is a non-selective COX inhibitor targeting cyclooxygenase (COX). Ketorolac may be administered orally, intramuscularly, or intravenously.

Dexamethasone refers to 9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one. Dexamethasone is a type of steroid medication. Dexamethasone may be administered orally, intravenously, intramuscularly, subcutaneously, or by Intraosseous infusion.

Prednisone refers to 17,21-dihydroxypregna-1,4-diene-3,11,20-trione. Prednisone is a synthetic corticosteroid drug. Prednisone may be administered orally, nasally, rectally, or intravenously.

The compounds may be in the form of a pharmaceutically acceptable salt thereof. The salt may include acid addition salts commonly used in pharmaceutical fields, for example, neurodegenerative disease. The acid addition salt may include those derived from inorganic acids, such as hydrochloric acid, bromic acid, sulfuric acid, sulfamic acid, phosphoric acid, or nitric acid, and those derived from organic acids, such as acetic acid, propionic acid, succinic acid, glycolic acid, stearic acid, citric acid, maleic acid, malonic acid, methanesulfonic acid, tartaric acid, malic acid, phenylacetic acid, glutamic acid, benzoic acid, salicylic acid, 2-acetoxybenzoic acid, fumaric acid, toluenesulfonic acid, oxalic acid, or trifluoroacetic acid. Further, the salt may include common metal salts, for example, those derived from metals, such as lithium, sodium, potassium, magnesium, or calcium. The acid addition salts or metal salts may be prepared by a general method.

The compound may be in the form of a stereoisomer thereof. The stereoisomer may be an enantiomer or a diastereomer. The compound may be a stereoisomerically pure form or a mixture of one or more stereoisomers, for example, a racemic mixture. Isolation of particular stereoisomers may be performed by any one of the general methods known in the art.

The compound may be in the form of a derivative thereof. The derivative may be a compound obtained by chemically modifying a part of the compound with other atoms or atomic groups.

The compound may be in the form of a solvate thereof. The solvate refers to a complex or aggregate formed by one or more molecules of a solute, i.e., the compound, or a pharmaceutically acceptable salt, stereoisomer, or derivative thereof, and one or more molecules of a solvent. The solvate may be a complex or aggregate formed with, for example, water, methanol, ethanol, isopropanol, or acetic acid.

The compound, pharmaceutically acceptable salt, stereoisomer, derivative, or solvate thereof, or combination thereof may induce expression of Parkin polypeptide. The Parkin polypeptide may be a polypeptide encoded by human PARK2 gene. The Parkin polypeptide may be a component of E3 ubiquitin ligase. The Parkin polypeptide may be a polypeptide having an amino acid sequence of GenBank Accession No. NP_004553 or a polypeptide encoded by a nucleotide sequence of GenBank Accession No. NM_004562 in humans. The Parkin polypeptide may be a polypeptide having an amino acid sequence of GenBank Accession No. NP_057903 or a polypeptide encoded by a nucleotide sequence of GenBank Accession No. NM_016694 in mice. The compound, pharmaceutically acceptable salt, stereoisomer, derivative, or solvate thereof, or combination thereof may induce transcription or translation of Parkin gene. For example, the compound, pharmaceutically acceptable salt, stereoisomer, derivative, or solvate thereof, or combination thereof may activate Parkin promoter to induce transcription of Parkin gene or to increase the intracellular level of Parkin polypeptide.

The neurodegenerative disease refers to pathological conditions characterized by degeneration of neuronal cells. The neurodegenerative disease includes those caused by degeneration of neuronal cells. The neurodegenerative disease may be one or more selected from Parkinson's disease, Alzheimer's disease, amyotrophic lateral sclerosis, Huntington's disease, fronto-temporal dementia, Lewy body dementia, vascular dementia, corticobasal degeneration, progressive supranuclear palsy, and multiple system atrophy.

The term “prevention” means all of the actions by which the occurrence or onset of neurodegenerative disease or symptom thereof is avoided, restrained or retarded by administration of the pharmaceutical composition, and the term “treatment” means all of the actions by which a neurodegenerative disease or symptom thereof is alleviated, or the progression slowed, to any degree (e.g., have taken a turn for the better or been modified favorably) by administration of the composition.

The pharmaceutical composition may further include a pharmaceutically acceptable carrier. In the pharmaceutical composition, the “pharmaceutically acceptable carrier” refers to a substance used in combination with an active ingredient to aid application of the active ingredient, and generally, refers to an inert material. The carrier includes general pharmaceutically acceptable excipients, additives, or diluents. The carrier includes one or more selected from the group consisting of, for example, a filler, a binder, a disintegrant, a buffering agent, a preservative, an antioxidant, a lubricant, a flavoring agent, a thickener, a coloring agent, an emulsifier, a suspension, a stabilizer, and an isotonic agent. The carrier may include, for example, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, or mineral oils.

The pharmaceutical composition may include the compound, pharmaceutically acceptable salt, stereoisomer, derivative, or solvate thereof, or combination thereof in a “therapeutically effective amount”. In the pharmaceutical composition, “therapeutically effective amount” refers to an amount sufficient to exhibit a therapeutic effect when administered to a subject in need of treatment. The effective amount may be determined depending on the severity of disease, a patient's age, body weight, health conditions, gender, and drug sensitivity, administration time, administration route, excretion rate, treatment period, and drugs blended with or co-administered with the composition, and other factors well known in the medical field. The “effective amount” may be about 0.01 mg to 10,000 mg, about 0.1 mg to 1000 mg, about 1 mg to 100 mg, about 0.01 mg to 1000 mg, about 0.01 mg to 100 mg, about 0.01 mg to 10 mg, or about 0.01 mg to 1 mg, based on the composition.

The administration dose of the pharmaceutical composition may be, for example, in the range of about 0.001 mg/kg to about 100 mg/kg, about 0.01 mg/kg to about 10 mg/kg, or about 0.1 mg/kg to about 1 mg/kg per adult once a day, several times a day, once every two days, once a week, once a month, or once a year.

The pharmaceutical composition may be administered via an oral route or a parenteral route including intravenous, intraperitoneal, subcutaneous, intramuscular, rectal and topical routes. Therefore, the composition may be formulated to various preparations such as a tablet, a capsule, an aqueous solution, a suspension, etc. The tablet for oral administration may be generally added with an excipient such as lactose, corn starch, etc., and a lubricant such as magnesium stearate, etc. In the capsule for oral administration, lactose and/or dry corn starch may be used as a diluent. If the aqueous suspension for oral administration is needed, the active ingredient may be bound with an emulsifier and/or a suspension. If necessary, a particular sweetening agent and/or flavoring agent may be added. For intraneuronal, intramuscular, intraperitoneal, subcutaneous, and intravenous administration, a sterile solution of the active ingredient may be generally prepared, and pH of the solution may be appropriately adjusted and buffered. For intravenous administration, a total concentration of the solute may be controlled to provide isotonicity to the formulation. The composition may be in the form of an aqueous solution containing a pharmaceutically acceptable carrier such as brine at a pH of 7.4. The solution may be administered to a patient's intramuscular or neuronal blood-stream by local bolus injection.

The pharmaceutical composition may further include one or more other active ingredients for preventing or treating neurodegenerative disease.

Another aspect provides a pharmaceutical composition for inducing expression of Parkin polypeptide, the composition including one or more compounds selected from the group consisting of deferasirox, hydrocortisone, ketorolac, dexamethasone, and prednisone, a pharmaceutically acceptable salt, stereoisomer, derivative, or solvate thereof, or a combination thereof.

The deferasirox, hydrocortisone, ketorolac, dexamethasone, prednisone, pharmaceutically acceptable salt, stereoisomer, derivative, solvate, Parkin polypeptide, induction of expression of Parkin polypeptide, and pharmaceutical composition are the same as described above.

The compound, pharmaceutically acceptable salt, stereoisomer, derivative, or solvate thereof, or combination thereof may have a cell protecting activity. The cell may be a neuronal cell, an epithelial cell, an immune cell, a germ cell, a stem cell, or a combination thereof. The neuronal cell may be a dopaminergic neuronal cell. The cell protecting activity may protect cells from oxidative stress. The compound, pharmaceutically acceptable salt, stereoisomer, derivative, or solvate thereof, or combination thereof may have an antioxidant activity. The compound, pharmaceutically acceptable salt, stereoisomer, derivative, or solvate thereof, or combination thereof may protect cells from oxidative stress by its antioxidant activity. The oxidative stress may be caused by, for example, hydrogen peroxide.

The deferasirox, hydrocortisone, and ketorolac may induce expression of Parkin polypeptide to prevent or treat neurodegenerative diseases which are caused by abnormal expression of Parkin polypeptide. Further, dexamethasone and prednisone may induce expression of Parkin polypeptide to prevent or treat neurodegenerative diseases which are caused by abnormal expression of Parkin polypeptide.

Further, the deferasirox, hydrocortisone, ketorolac, dexamethasone and prednisone may induce expression of Parkin polypeptide to prevent or treat cancer which is caused by abnormal expression of Parkin polypeptide. The cancer may be, for example, melanoma, breast cancer, colon cancer, lung cancer, neuroblastoma, or a combination thereof.

Still another aspect provides a method of preventing or treating neurodegenerative disease, the method including administering to a cell or a subject one or more compounds selected from the group consisting of deferasirox, hydrocortisone, ketorolac, dexamethasone, and prednisone, a pharmaceutically acceptable salt, stereoisomer, derivative, or solvate thereof, or a combination thereof.

The deferasirox, hydrocortisone, ketorolac, dexamethasone, prednisone, pharmaceutically acceptable salt, stereoisomer, derivative, solvate, cell, neurodegenerative disease, prevention, and treatment are the same as described above.

The subject may be a mammal, for example, a human, a cow, a horse, a pig, a dog, a sheep, a goat, a rat, a mouse, a rabbit or a cat.

The cell or subject may be a cell or a subject having neurodegenerative disease or at the risk of having neurodegenerative disease.

The administration may be oral or parenteral administration. The administration may be topical or systemic administration. The administration dose may be, for example, in the range of about 0.001 mg/kg to about 100 mg/kg, about 0.01 mg/kg to about 10 mg/kg, or about 0.1 mg/kg to about 1 mg/kg per adult once a day, several times a day, once every few days during a period of one day to 1 year. The administration may be performed by a method known in the art. The composition may be administered directly to a subject by any means such as oral, intravenous, intramuscular, oral, transdermal, mucosal, intranasal, intratracheal or subcutaneous administration. The administration may be topical or systemic administration. The administration may be performed topically into cerebral vessels.

Still another aspect provides a method of inducing expression of Parkin polypeptide in a cell or in a cell of a subject, the method including administering to the cell or the subject one or more compounds selected from the group consisting of deferasirox, hydrocortisone, ketorolac, dexamethasone, and prednisone, a pharmaceutically acceptable salt, stereoisomer, derivative, or solvate thereof, or a combination thereof.

The deferasirox, hydrocortisone, ketorolac, dexamethasone, prednisone, pharmaceutically acceptable salt, stereoisomer, derivative, solvate, cell, subject, administration, Parkin polypeptide, and induction of expression of Parkin polypeptide are the same as described above.

The compound, pharmaceutically acceptable salt, stereoisomer, derivative, or solvate thereof, or combination thereof may induce expression of Parkin polypeptide to protect cells from oxidative stress. The compound, pharmaceutically acceptable salt, stereoisomer, derivative, or solvate thereof, or combination thereof may induce expression of Parkin polypeptide to inhibit cell death.

According to one or more compounds selected from the group consisting of deferasirox, hydrocortisone, ketorolac, dexamethasone, and prednisone, a pharmaceutically acceptable salt, stereoisomer, derivative, or solvate thereof, or a combination thereof, and use thereof, Parkin expression of cells may be induced and oxidative stress of cells may be inhibited, thereby inhibiting cell death. The compound, pharmaceutically acceptable salt, stereoisomer, derivative, or solvate thereof, or combination thereof inhibits cell death, thereby efficiently preventing or treating neurodegenerative diseases.

All aspects of the methods are otherwise as described with respect to the pharmaceutical composition.

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items.

Hereinafter, the present invention will be described in more detail with reference to Examples. However, these Examples are for illustrative purposes only, and the invention is not intended to be limited by these Examples.

### EXAMPLE 1

Screening of Compounds Inducing Parkin Expression and Verification Thereof

1. Establishment of HEK-293T-Parkin Luciferase Cell Line

To identify compounds capable of inducing expression of Parkin protein at the promoter level, a pGL3-Parkin-Luc DNA construct was used (Cell Death and Differentiation, vol. 18, No. 5, p. 769-782, 2011.05 ). This construct was used to quantify the activity of human Parkin promoter by luminescence intensity due to a luciferase expression level. As a cell line, a human embryonic kidney cell line HEK-293T (ATCC), which maintains Parkin protein activity at a low level, was prepared.

To obtain more stable luciferase expression and drug response, puromycin marker DNA (Clontech) and pGL3-Parkin-Luc DNA were co-transfected into the prepared HEK-293T cells. Thereafter, 5 μg/ml of puromycin (Calbiochem) was added to the HEK-293T cells, and cultured at 37° C. and 5 % CO₂ for about 21 days. Every 3 days, the medium was replaced by a fresh medium containing 5 μg/ml of puromycin. Stable cell lines were established from single colonies of the cultured cell line. A cell line showing stable luciferase activity was obtained, and designated as HEK-Parkin-Luc, which was used to screen for compounds capable of activating the Parkin promoter. The HEK-Parkin-Luc cell line may be used for screening Parkin inducers and also for predicting Parkin promoter activity associated with various pathological and physiological changes.

2. Screening of Parkin Inducer

HEK-Parkin-Luc cell line of 1 was inoculated at a confluence of about 80% in a 96-well plate, and cultured at 37° C. and 5% CO₂ for about 24 hours. Thereafter, 10 μM of active compounds (in 0.1% DMSO) for screening was added to the cultured cells, and cultured at 37° C. and 5% CO₂ for about 24 hours. As the active compounds for screening, a library of a total of 1152 compounds was used (Selleck Chemicals).

Then, the cultured cells were washed with PBS, and a luminescent substrate (Promega) was added. Luminescence intensity of the cells was measured using a microplate luminometer (Berthold Technologies), and luciferase activity relative to a negative control of 0.1% DMSO was calculated.

Among a total of 1152 compounds, 44 compounds induced an increase in Parkin promoter activity of 1.5-foldor higher relative to the negative control. The screen was repeated three times using only the 44 compounds that showed an initial response, which resulted in 18 compounds showing a significant (P<0.001 ) increase in Parkin promoter activity of 1.5-foldor higher relative to the negative control. Among these 18 compounds were compounds targeting kinase such as Janus kinase (JAK) and phosphoinositide 3-kinase (PI3K), mTOR (Mammalian target of rapamycin), gamma-secretase, Histone deacetylase (HDAC), or proteasome.

3. Validation of Parkin Activator

Among the 44 compounds selected in the initial screen described in 2, 22 compounds were examined to determine whether they increased expression of Parkin protein when they are applied to an HEK 293T cell line.

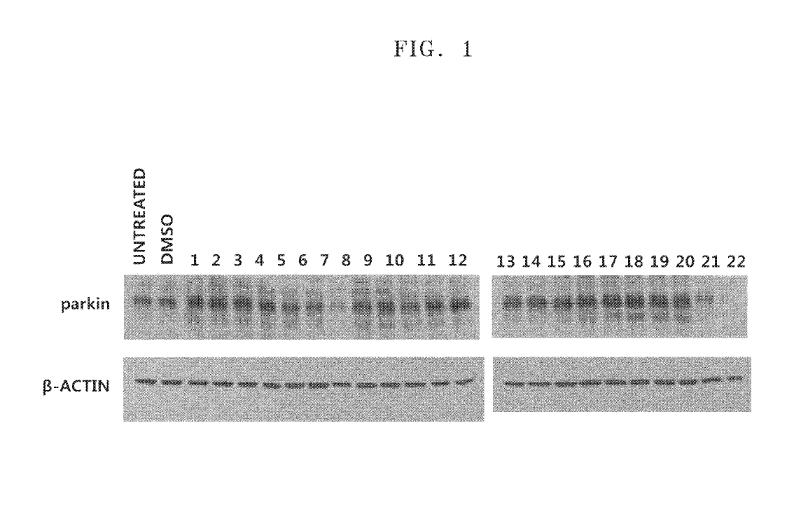

10 μM of each compound was added to the HEK 293T cells, and cultured at 37° C. and 5% CO₂ for about 48 hours. The cultured cells were collected, and proteins were obtained from the cells. Anti-Parkin protein antibody (Cell signaling) and anti-beta actin antibody (Sigma-Aldrich) were used to perform immunoblotting, and an image of the immunoblotting is shown in FIG. 1.

As shown in FIG. 1, the level of Parkin protein was increased in most cells treated with respective compounds, compared to a negative control. Therefore, the selected compounds were found to activate Parkin at promoter and protein levels in the same pattern.

4. Validation of Cell-Protecting Effect

Since Parkin protein is known to be an E3 ligase having a cell protecting effect, it was examined whether any of the 22 compounds selected in 2 were capable of protecting cells from oxidative stress.

A cytotoxicity-induced model by application of oxidative stress to the HEK 293T cell line was used. 10 μM of each of the 22 compounds was applied to the HEK cell line and cultured at 37° C. and 5% CO₂ for about 48 hours. H₂O₂ (Sigma-Aldrich, final concentration of 500 μM) was applied to the cultured cells and cultured at 37° C. and 5% CO₂ for about 90 minutes. Thereafter, cells were collected and cytotoxicity was examined by Alamar blue® assay (Thermo Fisher Scientific).

The level of H₂O₂-induced cytotoxicity in cells treated with each of the 22 compounds was compared with cells treated with DMSO alone. 16 compounds having a significant cytotoxicity inhibitory effect (P<0.05) against oxidative stress were selected.

5. Selection of Compounds Having Neuronal Cytotoxicity-Inhibiting Effect

Through the Parkin promoter luciferase screening, immunoblotting, and cytotoxicity test in 2 to 3, deferasirox, hydrocortisone, and ketorolac were selected as compounds having a Parkin-inducing activity. Therefore, these compounds were selected as candidates for having an inhibitory effect on neuronal cytotoxicity.

TABLE 1

| | Luciferase | Immuno- | Cell protecting | Target |
|---|---|---|---|---|
| Compound | activity | blotting | effect (%) | molecule |
| DMSO | 1 | 1 | 0 | |
| | (n7, | | (n = 6, | |
| | s.e. = 0.150) | | s.e. = 0.881) | |
| Deferasirox | 1.62 | 1.84 | 21.45 | others |
| | (n = 8, | | (n = 5, | |
| | s.e. = 0.046) | | s.e. = 4.35) | |
| Hydrocortisone | 1.52 | 1.11 | 35.53 | others |
| | (n = 8, | | (n = 7, | |
| | s.e. = 0.159) | | s.e. = 9.36) | |
| Ketorolac | 1.31 | 1.95 | 21.49 | COX |
| tromethamine | (n = 6, | | (n = 5, | |
| | s.e. = 0.038) | | s.e. = 6.18) | |

In Table 1, the values of luciferase activity and immunoblotting were expressed relative to DMSO, and the cell protecting effect (%) represents a cytotoxicity inhibition ratio, relative to DMSO. As the value of the cell protecting effect increased, cytotoxicity due to oxidative stress was more effectively inhibited. Further, “n” represents the number of experiments that were repeated, and “s.e.” represents standard error.

6. Effect of Hydrocortisone in Dopamine Neurodegeneration

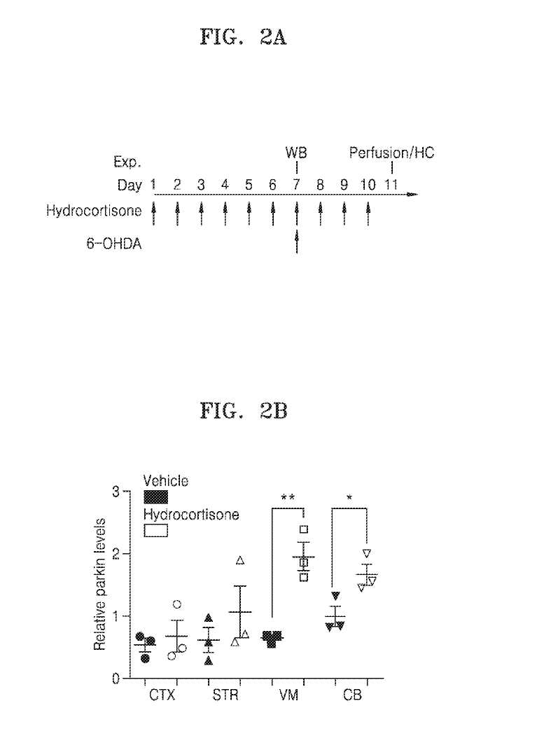

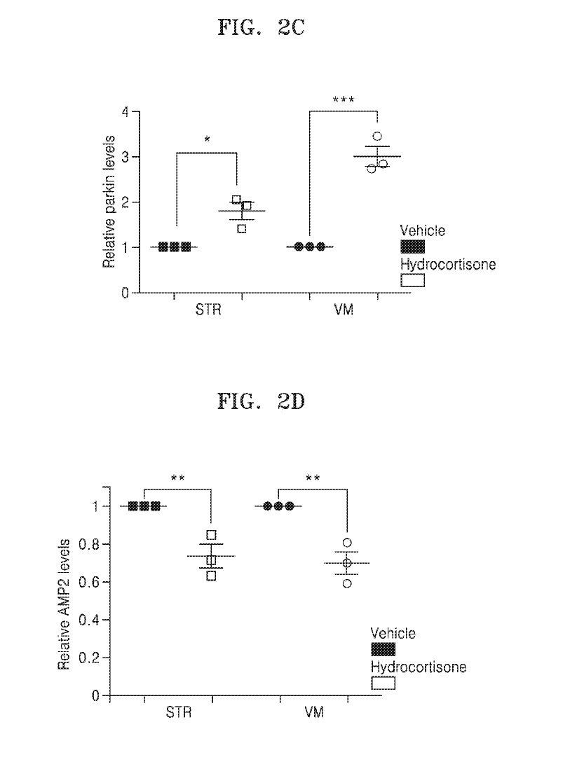

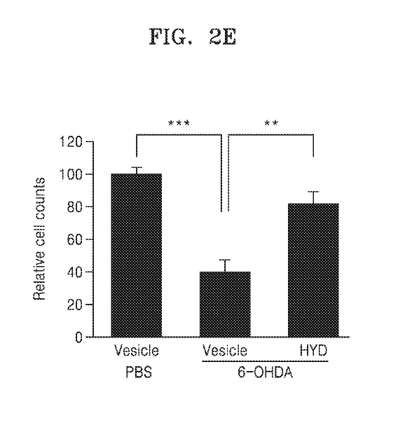

Hydrocortisone has been shown to penetrate into brain for its diverse physiological actions through glucocorticoid receptor. To ascertain whether hydrocortisone can increase parkin expression in vivo, we administered hydrocortisone for 7 consecutive days intraperitoneally to examine parkin expression in mouse brains (FIG. 2A).

Specifically, all animal experiments were approved by the Sungkyunkwan University Ethical Committee in accordance with international guidelines. Male C57BL/6J background mice were obtained from Orient (Suwon, Korea) and maintained at 12-h dark/light cycles in air-controlled rooms, with access to diet and water ad libitum. All efforts were made to minimize animal suffering, and to reduce the number of animals used.

Hydrocortisone was given to mice intraperitoneally. Hydrocortisone administration began on day 1 and continued for 11 days followed by stereological assessment of dopamine neuron counts. 6-OHDA intrastriatal injection was done on day 7. For stereotaxic injection of 6-hydroxy dopamine (6-OHDA, 8 ug), eight-week-old C57/BL6 mice treated either with hydrocortisone for 7 days or DMSO as a control were anesthetized with pentobarbital (60 mg/kg). An injection cannula (26.5 gauge) was applied stereotaxically into the striatum (anteroposterior, 0.5 mm from bregma; mediolateral, 2.0 mm; dorsoventral, 3.0 mm) unilaterally (applied into the right hemisphere). The infusion was performed at a rate of 0.2 μl/min, and 2μl of 6-OHDA (4 ug/ul in sterile PBS) was injected into each mouse. After the final injection, the injection cannula was maintained in the striatum for an additional 5 minutes for a complete absorption of the chemical and then slowly removed from the mouse brain. The head skin was closed by suturing and wound healing and recovery were monitored following surgery. For stereological analysis, animals were perfused and fixed intracardially with ice-cold PBS followed by 4% paraformaldehyde 4 days after intrastriatal 6-OHDA injection. The brain was removed and processed for immunohistochemistry. Mouse brain tissues harvested and homogenized in lysis buffer [10 mM Tris-HCl, pH 7.4, 150 mM NaCl, 5 mM EDTA, 0.5% Nonidet P-40, 10 mM Na-β-glycerophosphate, Phosphate Inhibitor Cocktail I and II (Sigma), and Complete Protease Inhibitor Mixture (Roche)], using a Diax 900 homogenizer. After homogenization, samples were rotated at 4° C. for 30 min for complete lysis, then the homogenate was centrifuged at 52,000 rpm for 20 min, and the resulting fractions were collected. Protein levels were quantified using the BCA kit (Pierce) with BSA standards and analyzed by immunoblot. Immunoblotting was performed with an antibody of interest with chemiluminescence visualization (Pierce).

The densitometric analyses of the bands were performed using ImageJ (NIH, http://rsb.info.nih.gov/ij/).

After scheduled treatments of hydrocortisone (0.4 mg/kg body weight, i.p. administration daily) in 6-OHDA intoxication models and control groups, animals were anesthetized with pentobarbital (50 mg/kg, intraperitoneal injection) and perfused with PBS followed by 4% paraformaldehyde (wt/vol in PBS). Brains were post-fixed with 4% paraformaldehyde overnight, and subsequently cryoprotected in 30% sucrose in PBS (wt/vol) overnight. Forty μm coronal sections were cut throughout the brain including substantia nigra and every 4th section was utilized for analysis. For tyrosine hydroxylase (TH), sections were incubated with a 1:1000 dilution of rabbit polyclonal anti-TH (Novus) followed by sequential incubations with biotinylated goat anti-rabbit IgG and streptavidin-conjugated horseradish peroxidase (HRP) according to the manufacturer's instructions (Vectastain ABC kit, Vector Laboratories, Burlingame, Calif.). 3,3-diaminobenzidine (DAB, cat# D4293, Sigma) was used as substrate for HRP to visualize TH positive cells. TH immunostained brain sections were counterstained with Nissl. Total numbers of TH-positive neurons in substantia nigra pars compacta were counted using the Optical Fractionator probe of Stereo Investigator software (MicroBrightfield, Williston, Vt.). Experimenters were blinded for treatments of mice during stereological counting.

In FIG. 2B, hydrocortisone administration led to an approximately two fold increase of parkin expression in the selected brain subregions (striatum, ventral midbrain, and cerebellum) when compared to the corresponding subregions of the vehicle treated control mice. In FIGS. 2C and 2D, hydrocortisone-stimulated parkin expression in the striatum and ventral midbrain corresponds to downregulation of parkin's toxic substrate AIMP2 by 20%.

Since parkin expression has been shown to be protective against dopaminergic neuronal damage in several Parkinson disease related animal models, we assessed neuroprotective effects of hydrocortisone in 6-hydroxydopamine (6-OHDA)-induced PD mouse models. 6-OHDA intrastriatal injection led to an approximate 60% loss of tyrosine hydroxylase (TH)-positive dopaminergic neurons in the substantia nigra pars compacta (FIG. 2E). In this regard, hydrocortisone pretreatment in mice provides markedly enhanced dopaminergic neuronal survival against 6-OHDA intoxication.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inducing expression of Parkin polypeptide in a cell, the method comprising administering to the cell or to a subject comprising the cell one or more compounds selected from the group consisting of deferasirox, hydrocortisone, ketorolac, dexamethasone, and prednisone, a pharmaceutically acceptable salt, stereoisomer, derivative, or solvate thereof, or a combination thereof.

2. The method of claim 1, wherein the compound, pharmaceutically acceptable salt, stereoisomer, derivative, or solvate thereof, or combination thereof induces expression of Parkin polypeptide to protect cells.

3. The method of claim 1, wherein the compound, pharmaceutically acceptable salt, stereoisomer, derivative, or solvate thereof, or combination thereof induces sufficient expression of Parkin polypeptide to inhibit cell death.

4. The method claim 1, wherein the cell is a neuronal cell, an epithelial cell, an immune cell, a germ cell, or a stem cell.

5. The method of claim 1, wherein the cell is in a subject.

6. The method of claim 5, wherein the subject is afflicted with a neurodegenerative disease.

7. The method of claim 6, wherein the neurodegenerative disease is Parkinson's disease, Alzheimer's disease, amyotrophic lateral sclerosis, Huntington's disease, frontotemporal dementia, Lewy body dementia, vascular dementia, corticobasal degeneration, progressive supranuclear palsy, multiple system atrophy, or combination thereof.

8. The method of claim 6, wherein the cell is a neuronal cell, and the administration of deferasirox, hydrocortisone, ketorolac, dexamethasone, prednisone, or pharmaceutically acceptable salt, stereoisomer, solvate, or derivative thereof, or combination thereof, inhibits death of the neuronal cell.

9. A method of preventing or treating neurodegenerative disease, the method comprising administering to a cell or a subject one or more compounds selected from the group consisting of deferasirox, hydrocortisone, ketorolac, dexamethasone, and prednisone, a pharmaceutically acceptable salt, stereoisomer, derivative, or solvate thereof, or a combination thereof.

10. The method of claim 9, wherein the cell or the subject is a cell or a subject having neurodegenerative disease or at the risk of having neurodegenerative disease.

11. The method of claim 9, wherein the administration is oral or parenteral administration.

12. The method of claim 9, wherein the neurodegenerative disease is Parkinson's disease, Alzheimer's disease, amyotrophic lateral sclerosis, Huntington's disease, frontotemporal dementia, Lewy body dementia, vascular dementia, corticobasal degeneration, progressive supranuclear palsy, multiple system atrophy, or combination thereof.
